(19) United States
(12) Patent Application Publication
(10) Pub. No.: US 2005/0232957 A1
(43) Pub. Date: Oct. 20, 2005

(54) Compositions and methods for moisturizing skin

(76) Inventor: Kenneth A. Katz, Philadelphia, PA (US)

Correspondence Address: NATH & ASSOCIATES, PLLC, Sixth Floor, 1030 15th Street, N.W., Washington, DC, 20005, US

(21) Appl. No.: 11/104,413

(22) Filed: Apr. 13, 2005

Related U.S. Application Data
(60) Provisional application No. 60/561,884, filed on Apr. 14, 2004.

(51) Int. Cl. A61K 31/60; A61K 31/415; A61K 7/00

(52) U.S. Cl. 424/401; 514/406; 514/165; 514/570; 514/569

(57) ABSTRACT

The inventive subject matter relates to novel topically applied specific and non-specific COX inhibitors, and topically applied aldosterone antagonists, and methods for producing increased skin moisturization. These compositions provide a new treatment option for dry skin.

This application claims the benefit of U.S. Provisional Patent Application No. 60/561,884, filed Apr. 14, 2004, the contents of which is hereby incorporated by reference in its entirety.

### BACKGROUND OF THE INVENTIVE SUBJECT MATTER

1. Field of Inventive Subject Matter

The present inventive subject matter relates to novel topically applied specific and non-specific COX inhibitors, and topically applied aldosterone antagonists, and methods for producing increased skin moisturization. These compositions provide a new treatment option for dry skin.

2. Background

Skin moisturizers are intended to increase water content of the outer layers of the skin. Moisture retention by current skin moisturizers is transient, as evaporation occurs rapidly, often within seconds or minutes, reducing the effectiveness of such products.

Epidermal absorption of lipid components of topical preparations are understood to be longer lived events; however, oil-based components of such products can contribute to undesirable characteristics and effects of formulations incorporating them. Many formulations rely on glycerol as a humectant; however, glycerol has only a short-term effect on increasing skin moisture levels. It is expected that the quality and effectiveness of moisturizing agents would be dramatically improved with the addition of compositions which enhance the retention of moisture in the skin.

Aquagenic wrinkling of the palms (“AWP”) has been observed in patients suffering from a confusing variety of diseases and disorders. For example, an important sign for the diagnosis of cystic fibrosis (“CF”) is an elevated concentration in chloride in the sweat. AWP is thought to be due to an increase in the water-binding capacity of keratins in the epidermis, mediated by increased salt concentrations. AWP has been observed in some CF patients, and also has been noted in patients with marasmus, a form of protein-energy malnutrition. Additionally, a case report has described an 18-year-old female who developed AWP one month after starting treatment with rofecoxib, a COX-2 inhibitor which is known to produce increased sodium concentrations in the kidney.

NSAIDs have been found to prevent the production of prostaglandins by inhibiting enzymes in the human arachidonic acid/prostaglandin pathway, including the enzyme cyclooxygenase (COX). The recent discovery of an inducible enzyme associated with inflammation (named “cyclooxygenase-2 (COX-2)” or “prostaglandin G/H synthase II”) provides a viable target of inhibition which more effectively reduces inflammation and produces fewer and less drastic side effects.

Some of the known compounds which selectively inhibit cyclooxygenase-2 have been described in U.S. Pat. Nos. 5,380,738, 5,344,991, 5,393,790, 5,434,178, 5,474,995, 5,510,368 and WO documents WO96/06840, WO96/03388, WO96/03387, WO96/25405, WO95/15316, WO94/15932, WO94/27980, WO95/00501, WO94/13635, WO94/20480, and WO94/26731.

Heretofore, the appearance of AWP has been considered an adverse and unusual symptom of disorders such as CF or marasmus, or an extremely rare side-effect of rofecoxib administration. Surprisingly, the present inventive subject matter provides new and effective compositions and methods for treating dry skin. Administering an inventive composition, and thereby increasing sodium chloride concentrations in the sweat and/or the water-binding capacity of keratins in the epidermis, moisturizes the skin and provides an effective treatment for dry skin and conditions associated with it, such as, for example, atopic dermatitis and various types of eczema.

### SUMMARY OF THE INVENTIVE SUBJECT MATTER

The present inventive subject matter relates to a topical moisturizing composition, comprising:
(a) a first moisturizing agent selected from the group consisting of a COX inhibitor, an aldosterone antagonist, and a mixture thereof; and
(b) a dermatologically acceptable carrier.

The inventive subject matter further relates to a method for moisturizing skin in a mammal, comprising topically administering to skin of a mammal in need thereof a therapeutically effective amount of a composition, comprising:
a) a first moisturizing agent selected from the group consisting of a COX inhibitor, an aldosterone antagonist, and a mixture thereof;
b) optionally, a second moisturizing agent; and
c) a dermatologically acceptable carrier.

The inventive subject matter also relates to a method for treating a skin disorder in a mammal, comprising topically administering to skin of a mammal in need thereof a therapeutically effective amount of a composition, comprising:
a) a first moisturizing agent selected from the group consisting of a COX inhibitor, an aldosterone antagonist, and a mixture thereof;
b) optionally, a second moisturizing agent; and
c) a dermatologically acceptable carrier.

In addition, the inventive subject matter relates to a method of treating or preventing dry skin in a mammal, comprising topically administering to skin of a mammal in need thereof a therapeutically effective amount of a composition, comprising:
a) a first moisturizing agent selected from the group consisting of a COX inhibitor, an aldosterone antagonist, and a mixture thereof;
b) optionally, a second moisturizing agent; and
c) a dermatologically acceptable carrier.

### DETAILED DESCRIPTION OF THE INVENTIVE SUBJECT MATTER

### DEFINITIONS

The term “therapeutically effective amount” as used herein refers to that amount of the inventive composition which will contribute to enhanced moisturization of the skin of a user.

The term “COX inhibitor” as used herein refers to a compound or composition which is able to inhibit a cyclooxygenase enzyme.

The term “cyclooxygenase-2 inhibitor” or “COX-2 inhibitor” as used herein refers to a compound or composition which is able to inhibit cyclooxygenase-2 without adverse inhibition of cyclooxygenase-1.

The term “aldosterone” as used herein refers to a mineralocorticoid that, among other effects, increases sodium resorption by cells in the kidney and the skin.

The term “aldosterone antagonists” as used herein refers to compositions which can inhibit the action of aldosterone and decrease sodium resorption by sweat glands.

### The Inventive Subject Matter

Cyclooxygenase is an enzyme-protein complex with a variety of biochemical actions. There are at least three primary COX isoenzymes, COX-1, COX-2, and COX-3. COX-1 is a constitutive enzyme, produced at a reasonably consistent level at all times. It plays an important role in, for example, gastrointestinal protection, kidney function, and the aggregation of blood platelets. COX-2 production is not constant; it varies depending on signals from various biochemical catalysts. For example, in the case of arthritis inflammation and pain, COX-2 responds to tissue damage by oxidizing arachidonic acid, creating prostaglandins which in turn produce local inflammation. COX-3 has been identified relatively recently (Chandrasekharan, et al., PNAS U.S.A., 99(21):13926-31 (2002)). In humans, COX-3 mRNA is expressed most abundantly in the cerebral cortex and heart tissues. COX-3 activity is selectively inhibited by analgesic/antipyretic drugs. It has been suggested that inhibition of COX-3 could represent a mechanism by which these drugs decrease pain and possibly fever.

Prostaglandins play a major role in the inflammatory process and the inhibition of prostaglandin production, especially production of PGG₂, PGH₂, and PGE₂, has been a common target of anti-inflammatory drug discovery. However, common non-steroidal anti-inflammatory drugs (hereinafter “NSAIDs”) that are active in reducing the prostaglandin-induced pain and swelling associated with the inflammation process are also active in affecting other prostaglandin-regulated processes not associated with the inflammation process.

NSAIDs have been found to prevent the production of prostaglandins by inhibiting enzymes in the human arachidonic acid/prostaglandin pathway, including the cyclooxygenase enzymes. Traditional non-steroidal anti-inflammatory drugs, such as aspirin, work by inhibiting both COX-1 and COX-2.

COX-2 is associated with inflammation and provides a viable target of inhibition which more effectively reduces inflammation and produces fewer and less drastic side effects. Thus, researchers have been motivated to develop selective COX-2 inhibitors to reduce inflammation and relieve pain without the gastrointestinal damage brought on by inhibiting COX-1. In addition, the current scientific understanding in the art suggests that COX-2 inhibition may serve an important function in promoting normal cell growth in the colon, pancreas, breast tissue, and other organ systems.

Some compounds which selectively inhibit cyclooxygenase-2 have been described in U.S. Pat. Nos. 5,380,738, 5,344,991, 5,393,790, 5,434,178, 5,474,995, 5,510,368 and WO documents WO96/06840, WO96/03388, WO96/03387, WO96/25405, WO95/15316, WO94/15932, WO94/27980, WO95/00501, WO94/13635, WO94/20480, and WO94/26731.

Drugs such as valdecoxib, celecoxib, and rofecoxib are intended to selectively inhibit COX-2 with minimal effect on COX-1.

Applicant expects that specific and non-specific COX inhibitors, aldosterone antagonists, and mixtures thereof produce an increase in cellular sodium resorption and a corresponding increase in the water-binding capacity of keratins in the epidermis, mediated by increased salt concentrations, producing an uptake of water.

Without being bound to a particular mechanism of action, it is expected that increased salt concentrations in the epidermis are caused by increased sodium chloride concentrations in the sweat and/or by increased sodium chloride resorption by keratinocytes. Regardless of the precise mechanism of action, such increased salt resorption produces a retention of moisture in the epidermis, which in the inventive subject matter is mediated in one of several ways, which are exemplified by the following:
1. By administration of nonspecific COX inhibitors such as ibuprofen or diclofenac.
2. By administration of specific COX inhibitors, such as rofecoxib or other COX-2 inhibitors.
3. By administration of antagonists to aldosterone, such as spironolactone or eplerenone.

Thus, the present inventive subject matter relates to a topical moisturizing composition, comprising:
(a) a first moisturizing agent selected from the group consisting of a COX inhibitor, an aldosterone antagonist, and a mixture thereof; and
(b) a dermatologically acceptable carrier.

In a preferred embodiment, said COX inhibitor is selected from the group consisting of a non-specific COX inhibitor, a COX-1 inhibitor, a COX-2 inhibitor, a COX-3 inhibitor, and a mixture thereof.

In another preferred embodiment, said COX inhibitor is a COX-2 inhibitor selected from the group consisting of valdecoxib, celecoxib, and rofecoxib.

In another preferred embodiment, said COX inhibitor is an NSAID. A preferred NSAID is selected from the group consisting of acetyl salicylic acid, salsalate, diflunisal, indomethacin, sulindac, tolmetin, etodolac, ketorolac, oxaprozin, diclofenac, benoxaprofen, carprofen, carprofen dechlorinated(2-(2-carbazolyl)propionic acid), carprofen(3-chlorocarbazole), chlorobenoxaprofen, 2,4-dichlorobenoxaprofen, cinoxacin, ciprofloxacin, decarboxy-ketoprofen, decarboxy-suprofen, decarboxy-benoxaprofen, decarboxy-tiaprofenic acid, enoxacin, fleroxacin, fleroxacin-oxide, flumequine, indoprofen, ketoprofen, lomelfloxacin, 2-methyl-4-oxo-2H-1,2-benzothiazine-1,1-dioxide, N-demethyl fleroxacin, nabumetone, nalidixic acid, naproxen, norfloxacin, ofloxacin, pefloxacin, pipemidic acid, piroxicam, suprofen, and tiaprofenic acid.

In an alternate aspect of the inventive subject matter, said COX inhibitor is a non-specific COX inhibitor.

In another aspect of the inventive subject matter, said aldosterone antagonist is selected from the group consisting of spironolactone and eplerenone.

In a preferred aspect of the inventive subject matter, said composition further comprises a second moisturizing agent. Non-limiting examples of specific humectants and/or moisturizers useful as said second moisturizing agent in preferred compositions include a moisturizing agent selected from the group consisting of C₃-C₆ diols and triols, glycerin, sorbitol, triacetin sorbitol, propylene glycol, dipropylene glycol, 1,3-butylene glycol, glucose, xylitol, maltitol, polyethylene glycol, hyaluronic acid, chondroitin sulfuric acid, polyoxyethylene methylglycoside, pyrrolidone carboxylate salts, and polyoxypropylene methylglycoside.

Without being limited thereto, the inventive compositions optionally incorporate other excipients, including carriers, diluents, solvents, surface active agents, also called surfactants, lubricants, plasticizers, disintegrants, inert substrate particles, matrix forming excipients, colorants, and miscellaneous materials such as buffers and adsorbents in order to prepare a particular medicated composition.

Examples of solvents useful in the present inventive compositions can be, but are not limited to, those selected from the group consisting of water, a volatile propellant, a C₁-C₆ fluid alkyl or branched alkyl alcohol, an aromatic alcohol, an ether of a sorbitol derivative, propylene carbonate, xylene, methylene chloride, ethylhexanediol, polysiloxanes, dimethyl ether, and mixtures thereof.

A wide variety of surfactants can be employed in the inventive compositions. These surfactants can include, for example, polyoxyethylene fatty ethers, polyoxyethylene fatty esters, fatty acids, sulfated fatty acids, phosphated fatty acids, sulfosuccinates, amphoteric surfactants, non-ionic poloxamers, non-ionic meroxapols, petroleum derivatives, aliphatic amines, polysiloxane derivatives, sorbitan fatty acid esters, pharmaceutically acceptable salts thereof, and mixtures thereof.

Other commonly known surfactants are further contemplated as within the scope of the present inventive subject matter. These other surfactants include, for example, those listed in the CTFA Cosmetic Ingredient Dictionary, Second Edition, The Cosmetic Toiletry and Fragrance Association, Inc., 1133 Fifteenth Street, N.W., Washington, D.C. 20005, 1977, the entire contents of which are hereby incorporated by reference.

Exemplary non-limiting lubricants which are of use as other excipients according to the present inventive subject matter may be selected from the group consisting of calcium stearate, canola oil, glyceryl palmitostearate, hydrogenated vegetable oil, magnesium oxide, mineral oil, poloxamer, polyethylene glycol, polyvinyl alcohol, sodium benzoate, sodium lauryl sulfate, sodium stearyl fumarate, stearic acid, sterilizable corn starch, talc, zinc stearate, and mixtures thereof.

Exemplary non-limiting plasticizers are selected from the group consisting of lanolin, mineral oil, petrolatum, benzyl phenylformate, chlorobutanol, glycerol, triacetin, diethyl phthalate, diethyl sebacate, triethyl citrate, crotonic acid, propylene glycol, castor oil, citric acid esters, butyl phthalate, dibutyl phthalate, dibutyl sebacate, polyethylene glycols, benzyl benzoate, glycerin, sorbitol, tributyl citrate, acetyltriethyl citrate, glyceryl triacetate, glyceryl tributarate, glyceryl diacetate, acetylated monoglycerides, other excipients, and mixtures thereof. As is evident, plasticizers may be hydrophobic as well as hydrophilic in nature.

Exemplary non-limiting disintegrants which are of use as other excipients according to the present inventive subject matter may be selected from the group consisting of alginic acid, carboxymethylcellulose, hydroxypropyl cellulose, microcrystalline cellulose, colloidal silicon dioxide, croscarmellose sodium, crospovidone, magnesium aluminum silicate, methylcellulose, polacrilin, povidone, sodium alginate, sodium starch glycolate, starch, and mixtures thereof.

In addition to the active therapeutic agent and the excipients listed above, the inventive preparations of the invention may additionally comprise other nonessential, optional ingredients known to a person of ordinary skill in the art as suitable for the inventive composition. For example, a topical preparation may optionally further include one or more preservatives well known in the art, such as benzoic acid, sorbic acid, methylparaben, propylparaben, ethylenediaminetetraacetic acid (EDTA), benzyl alcohol, phenoxyethanol, DMDM hydantoin, and imidazolidinyl urea. These preservatives may be present in amounts up to about 1% and preferably from about 0.05 to about 0.5% by weight of the inventive composition. Other additional optional ingredients may include thickeners and viscosity modifiers such as diethanolamide of a long chain fatty acid, fatty alcohols (i.e. cetearyl alcohol), sodium chloride, sodium sulfate, ethyl alcohol, hydroxyethyl cellulose, and Carbomer; coloring agents such as any of the FD&C or D&C dyes; hair oxidizing (bleaching) agents such as hydrogen peroxide, perborate salts, and persulfate salts; hair reducing agents such as the thioglycolates; perfumes; moisturizers, emollients; plasticizers; stabilizers; skin penetrating agents; and chelating agents such as disodium EDTA.

The present inventive compositions are optionally formed as an oil-in-water emulsion, i.e. an emulsion having an oil phase and an aqueous phase. Preferably, the oil phase of the emulsion comprises an oily material and an emulsifier to aid in formation of the emulsion. More preferably, the oil phase contains at least two emulsifiers. Non-limiting exemplary oily materials include mineral oil, petrolatum, petroleum derivatives, fatty acids, fatty acid derivatives, fatty alcohols, fatty alcohol derivatives, paraffins, and mixtures thereof.

To form an emulsion, at least one emulsifier is used. In a preferred embodiment, at least two emulsifiers are present in an oil phase to help form the emulsion. Preferred, non-limiting examples of emulsifiers used in the present inventive compositions include polyoxyethylene sorbitan fatty acid esters, sorbitan fatty acid esters, propylene glycol stearate, glyceryl monostearate, polyethylene glycol, fatty alcohols, polymeric ethylene oxide-propylene oxide block polymers, derivatives thereof, pharmaceutically acceptable salts thereof, and mixtures thereof. In a preferred embodiment, the emulsifiers used in the present inventive compositions are either naturally or synthetically prepared.

The present inventive compositions may further comprise several additional excipients commonly known to those of ordinary skill in the art as useful in topical compositions. Several non-limiting examples of such additional excipients include antioxidants, chelates, preservatives, emollients, humectants, fluid alkyl alcohols, thickening agents, pH modifier, and mixtures thereof.

Non-limiting examples of specific antioxidants useful in the present inventive compositions include ascorbic acid, fumaric acid, malic acid, alpha tocopherol, ascorbic acid palmitate, butylated hydroxyanisole, propyl gallate, sodium ascorbate, sodium metabisulfite, and mixtures thereof.

Non-limiting examples of specific preservatives useful in the present inventive compositions include methylparaben, benzalkonium chloride, propylparaben, benzoic acid, EDTA, phenolic acid, sorbic acid, benzyl alcohol, isopropyl alcohol, benzethonium chloride, bronopol, butylparaben, cetrimide, chlorhexidine, chlorobutanol, chlorocresol, cresol, ethylparaben, glycerol, imidurea, phenol, phenoxyethanol, phenylethyl alcohol, potassium sorbate, propylene glycol, sodium benzoate, sodium propionate, sorbic acid, thimerosol, and mixtures thereof.

Non-limiting examples of specific emollients useful in the present inventive compositions include myristyl lactate, isopropyl -palmitate, light liquid paraffin, cetearyl alcohol, lanolin, mineral oil, petrolatum, ceryl esters wax, cholesterol, glycerol, glycerol monostearate, isopropyl myristate, lecithin, and mixtures thereof.

Non-limiting examples of specific humectants useful in the present inventive compositions include glycerin, propylene glycol, sorbitol, and triacetin.

Non-limiting examples of specific fluid alkyl alcohols useful in the present inventive compositions include ethanol, isopropyl alcohol, octodecyl alcohol, propyl alcohol, butanol, and pentanol.

Non-limiting examples of specific thickening agents useful in the present inventive compositions include cetyl alcohol, Carbomers, acrylates/C10-30 alkyl acrylate crosspolymers, hydroxyethylcellulose, hydroxypropylcellulose, polyethylene oxide, and mixtures thereof.

The pH modifiers useful in the present inventive compositions include acids, bases, and mixtures thereof. Preferred non-limiting examples of pH modifiers in this regard include acetic acid, acetylsalicyclic acid, ascorbic acid, boric acid, carbonic acid, citric acid, formic acid, ethanesulfonic acid, fumaric acid, glycerophosphoric acid, hippuric acid, hydrochloric acid, maleic acid, methanesulfonic acid, nitrous acid, oxalic acid, phosphoric acid, saccharin, sorbic acid, sulfuric acid, thiosulfuric acid, undecylenic acid, ethanolamine, triethanolamine, sodium carbonate, sodium acetate, sodium hydrogen phosphate, sodium dihydrogen phosphate, sodium citrate, sodium bicarbonate, sodium hydroxide, and mixtures thereof.

The inventive compositions may also be packaged in a container suitable for storage and delivery of said composition.

The inventive compositions are particularly useful in topical formulations for treating a wide variety of dermatological diseases, disorders, or conditions in a mammal, especially diseases affecting mammalian tissue. Examples of such diseases include, but are not limited to, eczema, infantile eczema, psoriasis, scalp psoriasis, atopic dermatitis, dermatitis herpetiformis, contact dermatitis, seborrheic dermatitis, neurodermatitis, pruritis, fungal diseases, and intertrigo.

It is contemplated that the inventive compositions may also optionally incorporate any active therapeutic agent which is compatible with water. A non-limiting list of such materials includes the following: vitamins, anti-infectives, and antibiotics.

Examples of preferred active therapeutic agents useful in the present inventive compositions can be, but are not limited to, those selected from the group consisting of steroids, antifungal agents, antimicrobials, agents intended to further protect the skin, modify its appearance, or improve its rate of healing, and mixtures thereof.

In a preferred embodiment, said steroid is a corticosteroid that works to beneficially alter the appearance, metabolic or functional state, permeability, or health of a living organism. Corticosteroids are steroid hormones produced by the cortex of the adrenal gland. Preferred corticosteroids useful in the present inventive compositions include, but are not limited to, alclometasone dipropionate, amcinonide, beclamethasone dipropionate, betamethasone benzoate, betamethasone dipropionate, betamethasone valerate, budesonide, clobetasol propionate, clobetasone butyrate, cortisone acetate, desonide, desoximetasone, diflorasone diacetate, diflucortolone valerate, fluclorolone acetonide, flumethasone pivalate, fluocinolone acetonide, fluocinonide, fluocortin butyl, fluocortolone preparations, fluprednidene acetate, flurandrenolide, flurandrenolone, fluticasone propionate, halcinonide, halobetasol propionate, hydrocortisone, hydrocortisone acetate, hydrocortisone butyrate, hydrocortisone propionate, hydrocortisone valerate, methylprednisolone acetate, mometasone furoate, pramoxine hydrochloride, prednisone acetate, prednisone valerate, triamcinolone acetonide, and mixtures thereof.

In another preferred embodiment, said additional active therapeutic agent is an antifungal agent which can include, but is not limited to, those selected from the group consisting of imidazoles, hydroxy pyridones, triazoles, allyl amines, undecylenic acid derivatives, tolnaftate, haloprogin, pyridinethiones, cloquinol, and mixtures thereof.

Preferred antifungal agents useful in the present inventive compositions include, but are not limited to, amphotericin B, butoconazole nitrate, ciclopirox olamine, clindamycin, clioquinol, clotrimazole, econazole, econazole nitrate, fluconazole, flucytosine, griseofulvin, itraconazole, ketoconazole, miconazole, micronazole, naftifine, nystatin, omadine disulfide, sulconazole, terbinafine, terconazole, tioconazole, tolnaftate, triacetin, unecylenic acid, zinc pyrithione, and mixtures thereof.

In another preferred embodiment, said active therapeutic agent is an antimicrobial agent which can include, but is not limited to, those selected from the group consisting of amikacin, bacitracin, colistin, gentamicin, kanamycin, metronidazole, mupirocin, neomycin, netilmicin, polymyxin B, streptomycin, tobramycin, phenols and cresols such as 2,4-dichloro-sym-metaxylenol, parachlorometaxylenol, and parachlorometacresol, bisphenols such as hexachlorophene, dichlorophene, bithionol, triclosan, and fentichlor, salicylanilides such as 4′,5-dibromsalicylanilide, 3′,4′,5-trichlorosalicylanilide, 3′,4′,5-tribromosalicylanilide, and 3,5,dibromo-3′-trifluoromethyl-salicylanilide, carbanilides such as trichlorocarbanilde and 3-trifluoromethyl-4-4′-dichlorocarbanilide, quaternary ammonium compounds such as alkyl-dimethyl benzyl ammonium chloride, alkyl-trimethyl ammonium chloride, alkyl trimethyl ammonium bromide, cetyl-trimethyl ammonium bromide, B-phenoxyethyl-dimethyl-dodecyl ammonium bromide, p-tert-octylphenoxyethoxyethyl-dimethyl-benzyl ammonium chloride, tetradecyl-pyridinium bromide, cetyl pyridinium bromide, cetyl pyridinium chloride, di-(n-octyl)-dimethyl ammonium bromide, alkyl-isoquinolinium bromide, 1-(3-chloroallyl)-3-5-7-triaza-1-azoniaadamantane chloride, and chlorhexidine (1,6,di(N-p-chlorophenylguanidino)hexane), 2-bromo-2-nitropropan-1,3-diol, imidazonidyl urea, ethanol, isopropyl alcohol, and mixtures thereof.

In another preferred embodiment, said active therapeutic agent is a skin-conditioning agent. Preferably, the skin-conditioning agent is selected from the group consisting of hydrocarbon oils and waxes, silicones, fatty acid derivatives, cholesterol, cholesterol derivatives, di- and tri-glycerides, vegetable oils, vegetable oil derivatives, liquid nondigestible oils such as those described in Mattson, U.S. Pat. No. 3,600,186, and Jandacek et al., U.S. Pat. Nos. 4,005,195 and 4,005,196, all of which are herein incorporated by reference in their entirety, or blends of liquid digestible or nondigestible oils with solid polyol polyesters such as those described in Jandacek, U.S. Pat. No. 4,797,300, and Letton, U.S. Pat. Nos. 5,306,514, 5,306,516, and 5,306,515, all of which are herein incorporated by reference in their entirety, acetoglyceride esters, alkyl esters, alkenyl esters, lanolin and its derivatives, milk tri-glycerides, wax esters, beeswax derivatives, sterols, phospholipids, and mixtures thereof.

In another preferred embodiment, said active therapeutic agent is an UV absorber/sunscreen agent. Preferably, the UV absorber/sunscreen agent is selected from the group consisting of para-aminobenzoic acid and its derivatives (ethyl, isobutyl, glyceryl esters), p-dimethylaminobenzoic acid and its derivatives (ethyl, isobutyl, glyceryl esters), o-aminobenzoates and its derivatives (methyl, menthyl, phenyl, benzyl, phenylethyl, linaly, terpenyl, and cyclohexenyl esters), salicylates (amyl, phenyl, benzyl, menthyl, glyceryl, and dipropylene-glycol esters), cinnamic acid derivatives (menthyl and benzyl esters; alphphenyl cinnamonitrile; butly cinnamoyl pyruvate, 2-ethylhexyl p-methoxycinnamate, iso-amyl p-methoxycinnamate), dihydroxycinnamic acid derivatives (umbelliferone, methyl-umbelliferone, methylaceto-umbelliferone), trihydroxycinnamic acid derivatives (esculetin, methylesculetin, daphnetin), hydrocarbons (diphenylbutadiene, stilbene), dibenzalacetone, benzalacetophenone, naphthosulphonates (sodium salts of 2-naphthol-3,6-disulphonic acid and of 2-naphthol-6,8-disulphonic acid), organic benzophenone derivatives (2,4-dihydroxybenzophenone, 2,2′,4,4′-tetrahydroxybenzophenone, 2-hydroxy-4-methoxybenzophenone, 2-hydroxy-4-methoxybenzophenone-5-sulfonic acid, 2,2′-dihydroxy-4,4′-dimethoxybenzophenone, 2,2′-dihydroxy-4-methoxybenzophenone, disodium 2,2′-dihydroxy-4,4′-dimethoxy-5,5′-disulfobenzophenone), zinc oxide, titanium dioxide, and mixtures thereof.

Another preferred active material used in the composition of the present invention is a drug indicated for the treatment of fungal infections. Classes of drugs indicated for the treatment of fungal infections include synthetic triazole, ergosterol inhibitor, and polyene antifungal. Specific examples of drugs indicated for the treatment of fungal infections are itraconazole, ketoconazole, and amphotericin B.

Examples of vitamins that are available as active ingredients include, without limitation, vitamin A (retinol), vitamin D (cholecalciferol), vitamin E group (α-tocopherol and other tocopherols), vitamin K group (phylloquinones and menaquinones), thiamine (vitamin B₁), riboflavin (vitamin B₂), niacin, vitamin B₆ group, folic acid, vitamin B₁₂ (cobalamins), biotin, vitamin C (ascorbic acid), and mixtures thereof

Other active therapeutic agents commonly known as useful in the preparation of topical compositions are further contemplated as within the scope of the present inventive subject matter.

The optimal formulations of the inventive subject matter will be determined by one skilled in the art depending upon considerations such as the route of administration and desired dosage. Such formulations may influence the physical state, stability, rate of in vivo release, and rate of in vivo clearance of the therapeutic agents of the invention.

### METHODS OF THE PRESENT INVENTIVE SUBJECT MATTER

The inventive subject matter further relates to a method for moisturizing skin in a mammal, comprising topically administering to skin of a mammal in need thereof a therapeutically effective amount of a composition, comprising:
a) a first moisturizing agent selected from the group consisting of a COX inhibitor, an aldosterone antagonist, and a mixture thereof;
b) optionally, a second moisturizing agent; and
c) a dermatologically acceptable carrier.

The inventive subject matter also relates to a method for treating a skin disorder in a mammal, comprising topically administering to skin of a mammal in need thereof a therapeutically effective amount of a composition, comprising:
a) a first moisturizing agent selected from the group consisting of a COX inhibitor, an aldosterone antagonist, and a mixture thereof;
b) optionally, a second moisturizing agent; and
c) a dermatologically acceptable carrier.

In an aspect of the inventive subject matter, several specific skin disorders may be treated according to the inventive methods. Exemplary among these skin disorders are atopic dermatitis, eczema, icthyotic atopic eczema, subacute and chronic atopic eczema, dry skin, cracked skin, disturbed barrier function, acthyotic skin, xerosis, rough skin, psoriasis, ichthyosis, keratosis, keratoderma, corns, calluses, scales, nummular dermatitis, lichen simplex chronicus, pityriasis rosea, dandruff, acne, pruritus, age spots, lentigines, melasmas, wrinkles, warts, blemished skin, hyperpigmented skin, hyperkeratotic skin, inflammatory dermatoses, skin changes associated with aging, skin requiring cleansers, and combinations thereof.

In a preferred embodiment, the skin disorder to be treated or prevented according to the present inventive methods is dry skin. Said topical administration moisturizes said dry skin. The administration of the present inventive compositions to the skin moisturizes the skin. Accordingly, in another preferred embodiment, the inventive methods improve the moisture content of skin to which the present topical compositions are administered.

Further, the inventive subject matter relates to a method of treating or preventing dry skin in a mammal, comprising topically administering to skin of a mammal in need thereof a therapeutically effective amount of a composition, comprising:
a) a first moisturizing agent selected from the group consisting of a COX inhibitor, an aldosterone antagonist, and a mixture thereof;
b) optionally, a second moisturizing agent; and
c) a dermatologically acceptable carrier.

### Dosage

The inventive compositions may usually be given in two to four doses daily. The amount of active ingredient that may be combined with excipients and carrier materials to produce a single dosage form will vary depending upon the host treated, the degree of moisturization desired, the nature of any skin disorder to be treated, and the particular mode of administration.

It is to be understood, however, that a specific dose level for any particular subject will depend upon a variety of factors, including the activity of the specific compound employed; the age, body weight, general health, sex and diet of the patient; the time of administration; the rates of absorption and excretion; the combination of active agents; the severity of the particular disorder or condition being treated; and the form of administration. One of ordinary skill in the art would appreciate the variability of such factors and would be able to establish specific dose levels using no more than routine experimentation.

### EXAMPLES

The following examples are illustrative of the present inventive subject matter and are not intended to be limitations thereon. Unless otherwise indicated, all percentages are based upon 100% by weight of the final composition.

### Example 1

### Effect of the Inventive Compositions on Atopic Dermatitis

A patient presents for treatment of atopic dermatitis. An inventive composition is periodically administered to said patient several times per day over a course of treatment lasting from several days to several months. The patient experiences a reversal or elimination in the dry skin symptoms of atopic dermatitis and a retention of moisture in the epidermis. With continuing treatment, the patient continues to exhibit no symptoms of dry skin, no long term side effects of the treatment, and improved skin tone and elasticity.

The inventive subject matter being thus described, it will be obvious that the same may be modified or varied in many ways. Such modifications and variations are not to be regarded as a departure from the spirit and scope of the inventive subject matter and all such modifications and variations are intended to be included within the scope of the following claims.

1. A topical moisturizing composition, comprising:
(a) a first moisturizing agent selected from the group consisting of a COX inhibitor, an aldosterone antagonist, and a mixture thereof; and
(b) a dermatologically acceptable carrier.

2. The composition of claim 1, wherein said COX inhibitor is selected from the group consisting of a non-specific COX inhibitor, a COX-1 inhibitor, a COX-2 inhibitor, a COX-3 inhibitor, and a mixture thereof.

3. The composition of claim 2, wherein said COX inhibitor is a COX-2 inhibitor selected from the group consisting of valdecoxib, celecoxib, and rofecoxib.

4. The composition of claim 2, wherein said COX inhibitor is an NSAID.

5. The composition of claim 4, wherein said NSAID is selected from the group consisting of acetyl salicylic acid, salsalate, diflunisal, indomethacin, sulindac, tolmetin, etodolac, ketorolac, oxaprozin, diclofenac, benoxaprofen, carprofen, carprofen dechlorinated (2-(2-carbazolyl)propionic acid), carprofen (3-chlorocarbazole), chlorobenoxaprofen, 2,4-dichlorobenoxaprofen, cinoxacin, ciprofloxacin, decarboxy-ketoprofen, decarboxy-suprofen, decarboxy-benoxaprofen, decarboxy-tiaprofenic acid, enoxacin, fleroxacin, fleroxacin-oxide, flumequine, indoprofen, ketoprofen, lomelfloxacin, 2-methyl-4-oxo-2H-1,2-benzothiazine-1,1-dioxide, N-demethyl fleroxacin, nabumetone, nalidixic acid, naproxen, norfloxacin, ofloxacin, pefloxacin, pipemidic acid, piroxicam, suprofen, and tiaprofenic acid.

6. The composition of claim 2, wherein said COX inhibitor is a non-specific COX inhibitor.

7. The composition of claim 1, wherein said aldosterone antagonist is selected from the group consisting of spironolactone and eplerenone.

8. The composition of claim 1, further comprising a second moisturizing agent.

9. The composition of claim 8, wherein said second moisturizing agent is selected from the group consisting of C₃-C₆ diols and triols, glycerin, sorbitol, triacetin sorbitol, propylene glycol, dipropylene glycol, 1,3-butylene glycol, glucose, xylitol, maltitol, polyethylene glycol, hyaluronic acid, chondroitin sulfuric acid, polyoxyethylene methylglycoside, pyrrolidone carboxylate salts, and polyoxypropylene methylglycoside.

10. A method for moisturizing skin in a mammal, comprising topically administering to skin of a mammal in need thereof a therapeutically effective amount of a composition, comprising:
a) a first moisturizing agent selected from the group consisting of a COX inhibitor, an aldosterone antagonist, and a mixture thereof;
b) optionally, a second moisturizing agent; and
c) a dermatologically acceptable carrier.

11. A method for treating a skin disorder in a mammal, comprising topically administering to skin of a mammal in need thereof a therapeutically effective amount of a composition, comprising:
a) a first moisturizing agent selected from the group consisting of a COX inhibitor, an aldosterone antagonist, and a mixture thereof;
b) optionally, a second moisturizing agent; and
c) a dermatologically acceptable carrier.

12. The method of claim 11, wherein said skin disorder is selected from the group consisting of atopic dermatitis, eczema, icthyotic atopic eczema, subacute and chronic atopic eczema, dry skin, cracked skin, disturbed barrier function, acthyotic skin, xerosis, rough skin, psoriasis, ichthyosis, keratosis, keratoderma, corns, calluses, scales, nummular dermatitis, lichen simplex chronicus, pityriasis rosea, dandruff, acne, pruritus, age spots, lentigines, melasmas, wrinkles, warts, blemished skin, hyperpigmented skin, hyperkeratotic skin, inflammatory dermatoses, skin changes associated with aging, skin requiring cleansers, and combinations thereof.

13. The method of claim 11, wherein said topical administration moisturizes said skin.

14. The method of claim 11, wherein said method improves moisture content of said skin.

15. A method of treating or preventing dry skin in a mammal, comprising topically administering to skin of a mammal in need thereof a therapeutically effective amount of a composition, comprising:
a) a first moisturizing agent selected from the group consisting of a COX inhibitor, an aldosterone antagonist, and a mixture thereof;
b) optionally, a second moisturizing agent; and
c) a dermatologically acceptable carrier.
